(19) United States
(12) Patent Application Publication
(10) Pub. No.: US 2015/0206302 A1
(43) Pub. Date: Jul. 23, 2015

(54) SYSTEMS, METHODS AND COMPUTER READABLE STORAGE MEDIA STORING INSTRUCTIONS FOR INTEGRATING FLUOROSCOPY VENOGRAM AND MYOCARDIAL IMAGES

(71) Applicant: EMORY UNIVERSITY, Atlanta, GA (US)

(72) Inventor: Ji Chen, Johns Creek, GA (US)

(73) Assignee: EMORY UNIVERSITY, Atlanta, GA (US)

(21) Appl. No.: 14/423,747

(22) PCT Filed: Aug. 29, 2013
(86) PCT No.: PCT/US2013/057223

§ 371 Date: Feb. 25, 2015

Related U.S. Application Data
(60) Provisional application No. 61/694,933, filed on Aug. 30, 2012.

(51) Int. Cl. G06T 7/00; A61B 6/00; A61B 6/03

(52) U.S. Cl. CPC G06T 7/0012; A61B 6/037; A61B 6/463; A61B 6/487; A61B 6/503; A61B 6/541; G06T 2207/10108; G06T 2207/10121; G06T 2207/30021; G06T 2207/30048; G06T 2207/30101

(57) ABSTRACT

Systems, methods, and computer-readable storage media relate to generate an integrated image including fluoroscopy venogram and myocardial image with left-ventricular (LV) contraction sequence and scar distribution. The method may include processing myocardial image to determine LV systolic and diastolic dyssynchrony, the processing including generating one or more quantitative indices, the quantitative indices including myocardial scar distribution and contraction sequence; generating an integrated image including myocardial image and fluoroscopy venogram data, the integrated image including at least one rank of lead placement quality. The fluoroscopy venogram data may be either 2D fluoroscopy venogram or 3D LV venous anatomy reconstructed from the 2D fluoroscopy venogram.

### CROSS REFERENCE TO RELATED APPLICATION

This application claims priority to Provisional Application Ser. No. 61/694,933 filed Aug. 30, 2012, which is hereby incorporated by reference in its entirety.

### ACKNOWLEDGEMENT

This invention was made with government support under Grant No. IR01HL094438, awarded by the National Institutes of Health. The government has certain rights in the invention.

### COPYRIGHT NOTICE

A portion of the disclosure of this patent document contains material which is subject to copyright protection. The copyright owner has no objection to the facsimile reproduction by any one of the patent document or patent disclosure, as it appears in the Patent and Trademark Office patent file or records, but otherwise reserves all copyrights whatsoever.

### BACKGROUND

Heart failure remains a major concern in developed countries. About 5.7 million people in the U.S. have heart failure, and it results in about 3000,000 deaths each year. One treatment option for patients with symptomatic heart failure (HF) resulting from systolic dysfunction is cardiac resynchronization therapy (CRT). CRT is achieved by simultaneously pacing both the left and right ventricles in a synchronized manner. A conventional CRT pacing device (also called a “biventricular pacemaker”) is an electronic, battery-powered device that is surgically implanted under the skin. The device includes two or three lead wires that are positioned in the heart to beat in a more balanced way.

Numerous clinical investigations have demonstrated that CRT can improve clinical status, functional capacity, and survival in select patients with ventricular dyssynchrony. One factor that affects the mechanical resynchronization with CRT is non-optimal implementation/application of the bi-ventricular pacing device. It has been shown that 1) the LV lead should NOT be placed within a region with myocardial scar where the electrical stimulation cannot be appropriately delivered and 2) the LV lead should be placed within a region with delayed mechanical activation so that the electrical stimulation can make the region contract earlier. However, information about viable myocardium and delayed contraction is not currently available to a practitioner.

### SUMMARY

Echocardiography (cardiac ultrasound), CT, and MRI have been used for optimizing LV lead placement. However, echocardiography requires an additional imaging procedure to assess both mechanical activation and scar, CT has not shown to be effective in optimizing placement of the lead, and MRI is time-consuming and complicated. Thus, there is a need for imaging to optimal lead placement location.

This disclosure generally relates to methods, systems, and computer readable storage media that include instructions generating an integrated image that includes fluoroscopy venogram and myocardial image with left-ventricular contraction sequence and scar distribution

In some embodiments, the method may include processing myocardial image to determine LV systolic and diastolic function (or dyssynchrony), the processing including generating myocardial scar distribution; and generating an integrated image including myocardial image and fluoroscopy venogram data, the integrated image including at least one rank of lead placement quality.

In some embodiments, the generating may include segmenting the myocardial image into a plurality of segments, and determining lead placement quality of each segment based on location, scar distribution, and contraction sequence. The quality may include a plurality of ranks, the ranks including qualitative and/or numerical ranks. The ranks may include “optimal,” “next optimal,” “not recommended.”

In some embodiments, the generating includes segmenting the myocardial image data, the segmenting including dividing the myocardial image data into at least three segments. The segments may include apex, mid and base segments. In some embodiments, the segments may include at least six segments. In certain embodiments, the segments may include thirteen segments.

In some embodiments, the method may include displaying the integrated image with the lead placement quality. In some embodiments, the method may include transmitting the integrated image to a cardiac interventional system. In some embodiments, the method may include receiving post-processed myocardial image data. In some embodiments, the method may further comprise processing quantifies myocardial image data. In some embodiments, the myocardial image data may be a SPECT image data.

In some embodiments, the processing may include determining samples of the image data; and normalize values of the samples according size of the LV myocardium. In some embodiments, the processing may include determining regional maximum counts and respective locations along one or more angles that are perpendicular to a constructed LV mid-surface; and constructing mid-surface of the LV mid surface using the locations of the regional maximum counts.

In some embodiments, the disclosure may relate to a method of generating an integrated image, the method may include: processing myocardial image to determine pre-assessment information, the pre-assessment information including one or more quantitative indices related to regional function and scar for one or more myocardial segments and lead placement quality; processing 2D fluoroscopy venogram data to determine 3D venous anatomy; and generating an integrated image including the myocardial image and the 3D venous anatomy. The integrated image may indicate the pre-assessment information.

In some embodiments, the generating may also include aligning of 3D venous anatomy to the myocardial image so that spatial positions of the 3D venous anatomy and the myocardial image data correspond to same spatial coordinates. The generating may also include registering the 3D venous anatomy and epicardial surface provided in the myocardial image. The registering including refining a position of the 3D venous anatomy with respect to the myocardial image.

In some embodiments, the generating may include overlaying or fusing the 3D venous anatomy onto the epicardial surface using the spatial position of the 3D venous anatomy determined in the registering.

In some embodiments, the disclosure may relate to a computer-readable storage medium storing instructions for generating an integrated image. The instructions may include quantifying SPECT image data to determine LV systolic and diastolic function (or dyssynchrony), the quantifying including generating myocardial scar distribution; and generating an integrated image including SPECT image data and fluoroscopy venogram data, the integrated image including at least one rank of lead placement quality.

In some embodiments, the disclosure may relate to a system for generating an integrating image. The system may include an image quantifier configured to quantify SPECT image data to determine LV systolic and diastolic dyssynchrony, the quantifying including generating myocardial scar distribution; and an integrated image generated configured to generate an integrated image including SPECT image data and fluoroscopy venogram data, the integrated image including at least one rank of lead placement quality.

Additional advantages of the disclosure will be series forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 illustrates an example of a CRT device implanted in a heart of a patient.

FIG. 2 illustrates a method of generating an integrated image, according to embodiments.

FIG. 3 illustrates a method according to embodiments for processing raw or partially processed image data.

FIG. 4A illustrates a method according to embodiments for quantifying the image data.

FIG. 4B illustrates a method of processing each temporal frame according to embodiments.

FIG. 5 shows an example of the spherical and cylindrical sampling.

FIG. 6 shows an example of myocardial perfusion images with left-ventricular contraction sequence and scar distribution for optimal LV lead placement in CRT therapy.

FIG. 7A illustrates a method according to embodiments for generating the images.

FIG. 7B illustrates a method according to embodiments for integrating the myocardial image and 3D venous anatomy.

FIG. 8 shows an example of an integrated image.

FIG. 9 shows an example of a system according to embodiments.

FIGS. 10-17 show examples of diagrams illustrating operation of an user interface.

FIGS. 18A-D show results of patient image data processed using the user interface.

### DETAILED DESCRIPTION OF THE EMBODIMENTS

The following description, numerous specific details are series forth such as examples of specific components, devices, methods, etc., in order to provide an understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

This disclosure generally relates to methods, systems, and computer readable storage media that generate fluoroscopy venogram and myocardial images with left-ventricular contraction sequence and scar distribution. The image may include at least lead placement quality rank. Thus, the image may optimize the placement of the lead.

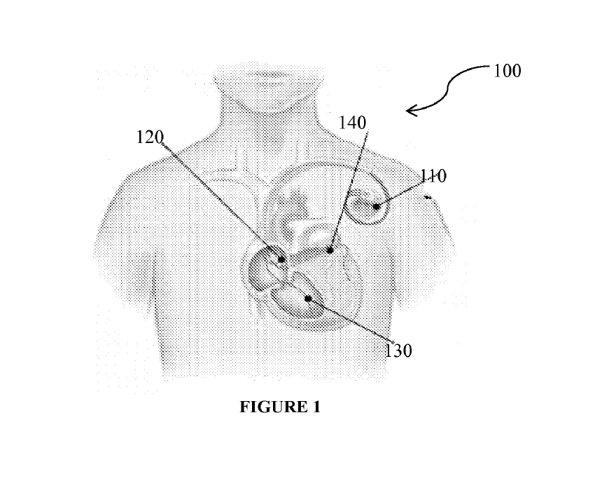

The generated image is described with respect to planning of or determining an optimal lead placement location before or during a CRT procedure, such as an implantation of a CRT pacing device (e.g., a biventricular pacemaker or a combination of a CRT and implantable cardiac defibrillator (ICD) device), respectively. An example of a CRT device implanted in a heart of a patient is shown in FIG. 1. As shown in FIG. 1, a CRT device 100 may include a pulse generator 110 that houses a battery and a computer connected to leads. The CRT device may include 2-3 leads positioned in the right atrium, right ventricle and left ventricle (via the coronary sinus vein). The device 100 shown in FIG. 1 includes leads 120, 130, 140 positioned in the right atrium, right ventricle and left ventricle, respectively. However, it should be understood that the disclosure is not limited to preplanning a CRT procedure and may be used other purposes. For example, the disclosure may be used in other medical intervention procedures planning, such as, for example, atrial fibrillation procedure planning, or atrial flutter procedure planning.

The disclosed methods, systems, and computer-readable media according to embodiments are discussed with respect to myocardial image data, more specifically, myocardial perfusion image data, acquired by a single-photon emission computed tomography (SPECT) system. It will be understood that the disclosed methods, systems, and computer-readable media may be applied to and/or include myocardial image data acquired from other imaging modalities, for example, those that have a capability of obtaining and/or generating 3D left-ventricular myocardial images with identifiable inter-ventricular grooves. The other imaging modalities may include but is not limited to positron emission tomography (PET), magnetic resonance imaging (MRI), cardiac computed tomography (CT), and echocardiographic systems.

The disclosed methods, systems, and computer-readable media according to embodiments are capable of being used intraoperatively to generate the integrated images using either 2D or 3D venous anatomy. The disclosed methods, systems, and computer-readable media according to embodiments thus address the deficiencies of mainstream CRT implantation.

Currently, mainstream CRT implantation generally uses fluoroscopy venograms to guide lead placement. Because fluoroscopy venograms only provide vessel information, the implanters can misplace the leads in regions with scar or without late contraction, deteriorating left-ventricular synchrony and causing non-response to CRT. Moreover, even though the myocardial information becomes available to the implanters, it can be difficult to correspond venous sites on the fluoroscopy venograms to myocardial regions. In the TARGET trial, LV leads were non-optimally placed in 37% of the patients with guided implantation, even if the implanters tried to place the leads into the target regions given by echocardiography pre implantation. Consequently, the response rate in the guided group was 70%, marginally better than the current practice. See, e.g., J Am Coll Cardiol 2012;59:1509-18.

### METHODS & GENERATED IMAGES

The methods of the disclosure are not limited to the steps described herein. The steps may be individually modified or omitted, as well as additional steps may be added.

Unless stated otherwise as apparent from the following discussion, it will be appreciated that terms such as “identifying,” “receiving,” “integrating,” “filtering,” “combining,” “reconstructing,” “segmenting,” “generating,” “registering,” “determining,” “obtaining,” “processing,” “computing,” “selecting,” “estimating,” “detecting,” “tracking,” “calculating,” “aligning” “fusing,” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

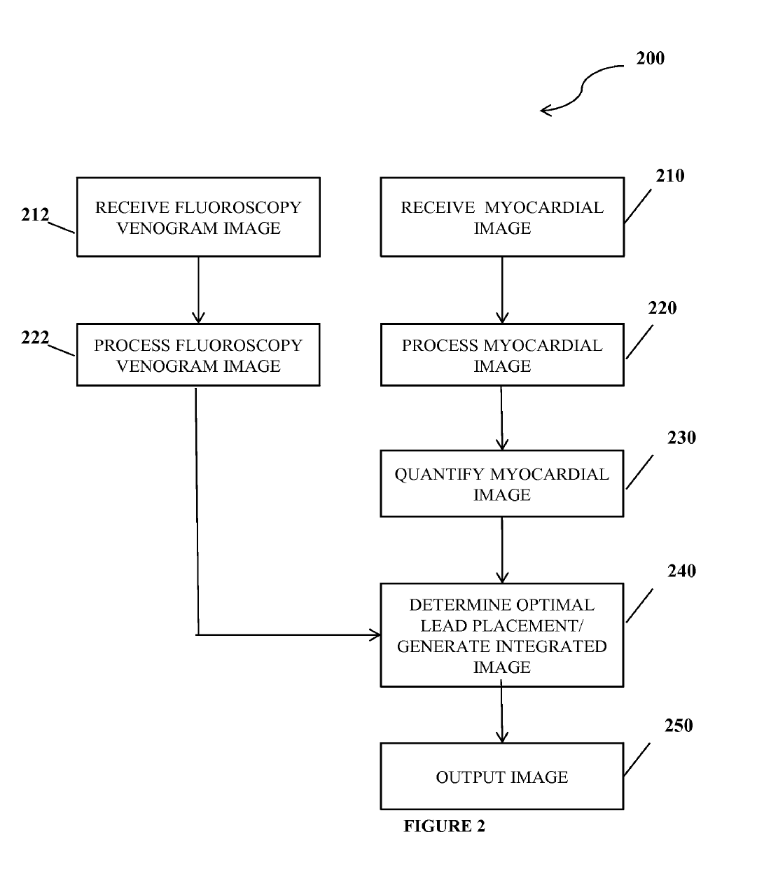

FIG. 2 illustrates a method 200 according to embodiments to generate an image that includes fluoroscopic venogram and myocardial images.

### Receiving

In some embodiments, the method 200 may include a step 210 of receiving (also referred to as “first”) image data. The image data may include myocardial image data. The first image data may be a heart of a patient.

In some embodiments, the myocardial image data may be received from an image acquisition device or from a data storage device. The image acquisition device may include any imaging modality, for example, any modality capable of obtaining and generating 3D left-ventricular myocardial images with identifiable inter-ventricular grooves. The imaging modality may include but is not limited to SPECT system, PET system, magnetic resonance imaging (MRI) system, cardiac CT systems, and echocardiography. For example, the myocardial image data may include myocardial perfusion image data (also referred to as “SPECT” image data) received and/or acquired from a SPECT system. The myocardial image data may also be myocardial image data obtained from PET, MRI, cardiac CT, and/or echocardiography systems.

In some embodiments, the myocardial image data may be in a Digital Imaging and Communications in Medicine (DICOM) format. The image data may include header and image data. The header may include image information. The image information may include information regarding the scan. The image information may include but is not limited to pixel counts, number of frames, dimensions of the image, data resolution, and image size. The image data may be raw data or processed image data.

In some embodiments, the method 200 may include a step 212 of receiving (also referred to as “second”) image data. The image data may include fluoroscopy venogram (also referred to as “fluoroscopic venogram”) image data. The image data may be of the heart of the same patient received in step 210.

The fluoroscopy venogram image data may be received from an image acquisition device or from a data storage device. In some embodiments, the fluoroscopy venogram image data may be received and/or acquired from any fluoroscopic imaging system capable of performing venography studies.

In some embodiments, the fluoroscopy venogram image data may be in a Digital Imaging and Communications in Medicine (DICOM) format. The image data may include header and image data. The header may include image information. The image information may include information regarding the scan. The image information may include but is not limited to pixel counts, acquisition angles, distance between the patient and the detector, distance between the source and the detector, number of frames, dimensions of the image, data resolution, and image size. The image data may be raw data or processed image data.

### Processing

In some embodiments, the received myocardial image data in step 210 may be raw image data. In some embodiments, the raw data may be raw image data of gated myocardial perfusion imaging (MPI), for example, acquired by single-photon emission computed tomography (SPECT). In some embodiments, the method 200 may include a step 220 of processing the myocardial image data. In other embodiments, the received myocardial image data may be post-processed image data.

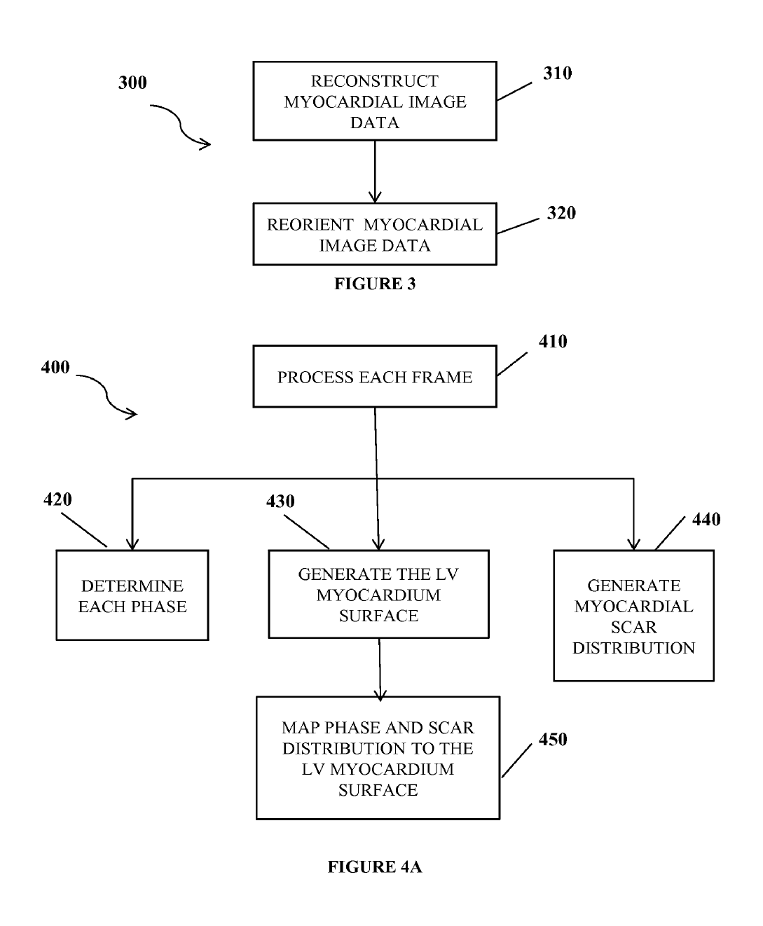

FIG. 3 shows a method 300 for processing the myocardial image data. In some embodiments, the processing step 220 may include one, some or all of the steps of method 300. In other embodiments, the processing step 220 may be based on any processing techniques.

As shown in in FIG. 3, the method 300 may include a step of reconstructing 310 the image data. The reconstructing step 310 may include identifying the left-ventricular (LV) myocardium in the image data. The reconstructing step 310 can process the raw data to generate tomographic images. The reconstructing step 310 may use ordered-subset expectation maximization (OSEM) techniques. The reconstructing step 310 may further include removing the noise from the generated tomographic images. In some embodiments, the noise may be removed by applying a Butterworth low-pass filter to images. In some embodiments, the parameters used in the Butterworth filter may include order=10, cutoff frequency=0.4 cycles/cm for the summed image, and cutoff frequency=0.35 cycles/cm for the gated image. The reconstructing step 310 may further include generating transaxial images of the LV myocardium.

In some embodiments, the reconstructing step 310 may be omitted. In some embodiments, the processing step may alternatively include receiving reconstructed image data or transaxial images, for example, from a SPECT system.

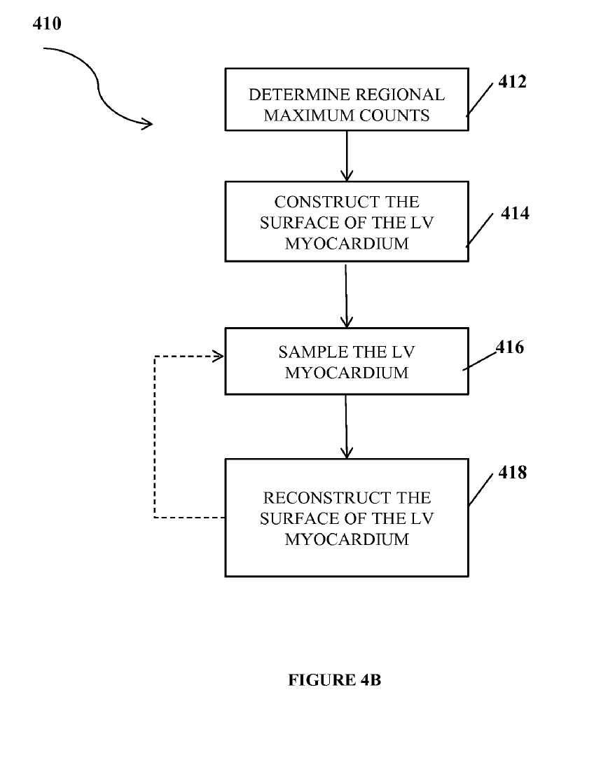

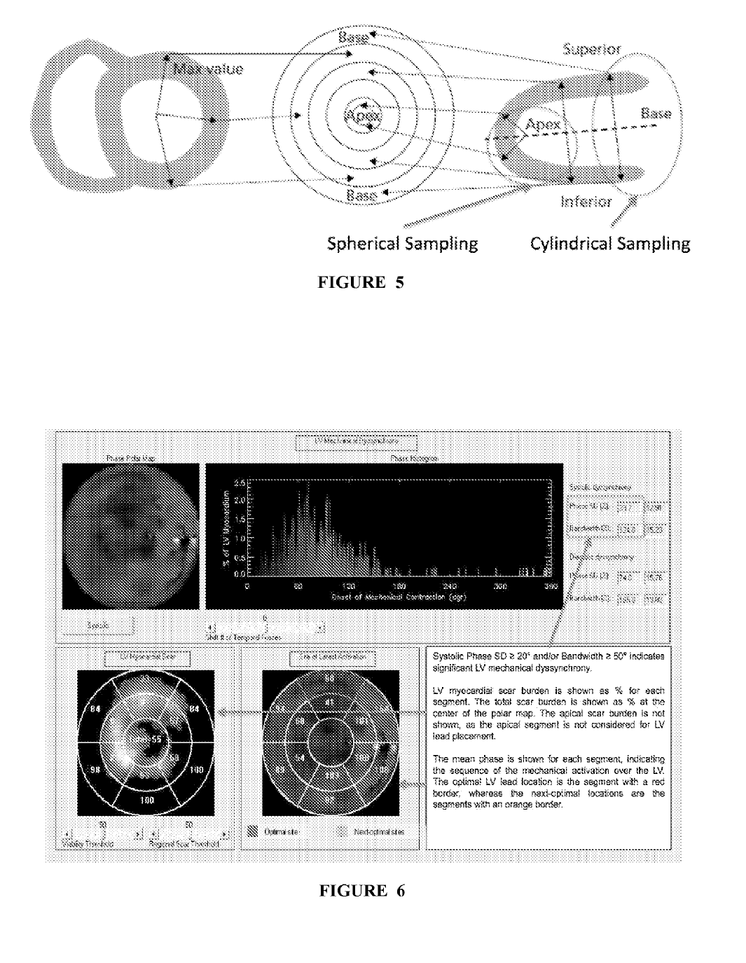

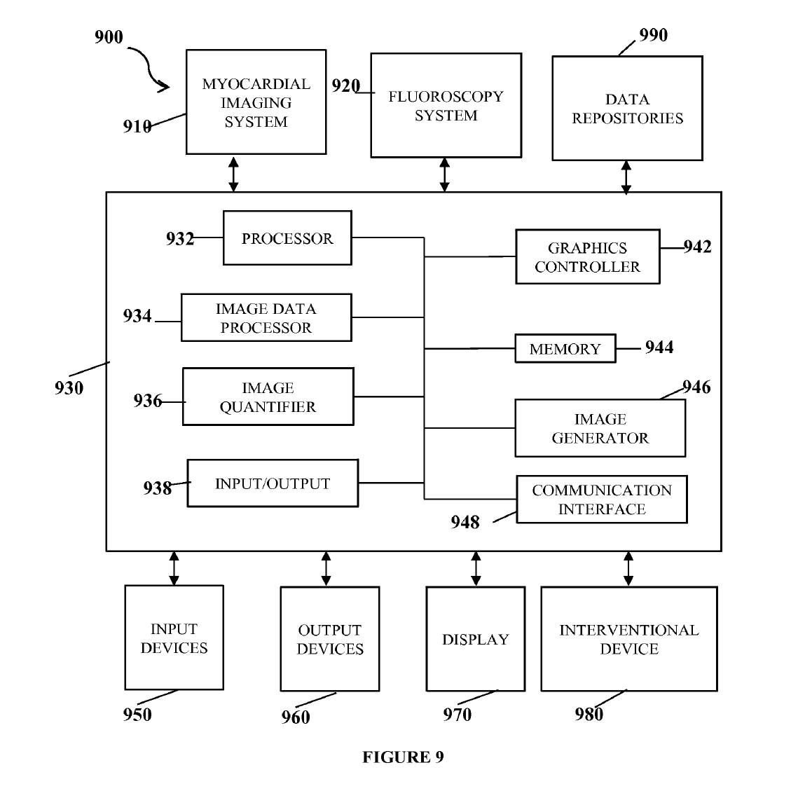

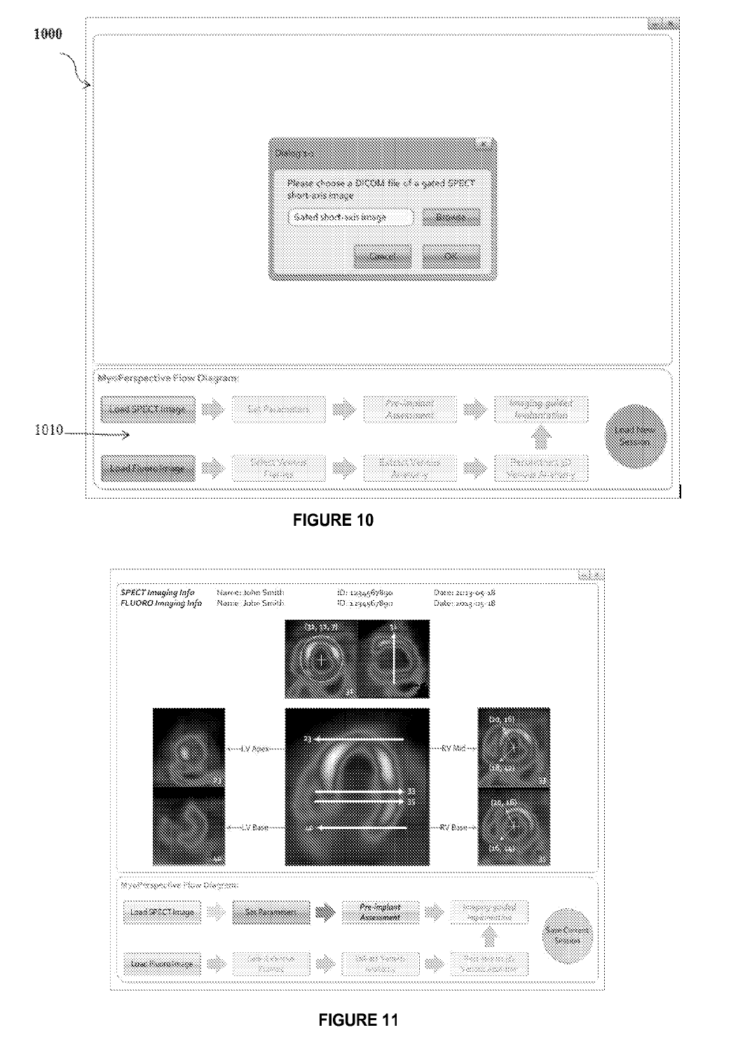

In some embodiments, the processing step 300 may include a step of reorienting 320 the image data. The reorienting step 320 may include a step of identifying the center of the left ventricle and the LV myocardium. In some embodiments, the step of identifying may include identifying the center of the left ventricle on the transaxial image and the sagittal image. In some embodiments, the center of the left ventricle may be identified by a line from the center towards the LV apex on the images. In some embodiments, the short-axis and the long-axis images of the LV myocardium may be optionally generated and displayed. In some embodiments, the step of identifying may include identifying the region of interest (ROI) for the LV myocardium. The ROI for the LV myocardium may be identified by a circle on the short-axis image. The center of the circle should approximate the center of the left ventricle and the circle should include the entire LV myocardium. The center slice of the left ventricle may be identified on the vertical long-axis image. On the horizontal long-axis image two pairs of lines may be identified to indicate the apex and base of the left and right ventricles, respectively. The center and radius of the circle over the left ventricle and the apex and base lines of the left ventricle can be used in the sampling method, for example, the sampling method shown and described with respect to FIG. 4B. The apex and base lines of the right ventricle and the points of the anterior and posterior interventricular grooves can be later used to define the anterior and posterior interventricular grooves in the integration step, for example, the landmark registration step shown and described with respect to FIG. 7B. The identifying steps may be repeated for each temporal frame.

In some embodiments, the reorienting step 320 may further include translating and rotating the images to generate short-axis images of the LV myocardium with region of interest. The translating and rotating may be according to any technique. The translating and rotating may be according to the starting point of the line provided on the transaxial image (the center of the left ventricle) and the angles of lines provided on the transaxial and sagittal images.

In some embodiments, the method 200 may include a step 222 of processing the fluoroscopy venogram image data. In some embodiments, the fluoroscopy venogram image data may be 2D fluoroscopy venogram image data. In some embodiments, the processing step 222 may process the 2D fluoroscopy venogram image data to generate 3D venous anatomy. In other embodiments, the method 200 may omit this step.

In some embodiments, the processing step 222 may include the processing 2D fluoroscopy venogram image data to reconstruct 3D anatomy. The processing 222 may include and/or be based on any reconstruction technique. In some embodiments, the processing 222 may include: 1) back projecting the identified venous anatomy on the fluoroscopy venograms along the direction of the fluoroscopy imaging; 2) find the middle point of each pair of the back-projection lines; and 3) connect all middle points to construct a 3D venous anatomy.

### Quantifying Myocardial Image Data

In some embodiments, the method 200 may include a step 230 of quantifying the myocardial image data to determine at least one quantitative index related to heart function (e.g., regional function and scar). The one or more quantitative indices may include but are not limited to at least the LV systolic and/or diastolic function, LV systolic and/or diastolic dyssynchrony, scar burden, scar distribution, or contraction sequence (also referred to as “phase”), as well as any combination thereof The quantitative indices may be included and/or a part of the pre-assessment information.

In some embodiments, the step of quantifying may include processing each temporal frame of the short-axis images of the LV myocardium 3D using an adaptive sampling function, for example, as shown in FIG. 4B.

FIG. 4A shows a method 400 of quantifying the myocardial image data to determine at least one of the quantitative indices, for example, phase and scar distribution of the LV myocardium. The method 400 may include a step of processing 410 each temporal frame of the LV myocardium to determine the pixel values of the samples and location of the samples. The processing step 410 may include applying an adaptive sample function according to embodiments, for example, as shown in FIG. 4B. It will be understood that the processing step 410 may be performed by applying other sampling techniques.

According to embodiments, the processing may normalize the sample values according to the size of the LV myocardium. The processing can also reduce the impact of changing LV size during the cardiac cycle. It can thereby improve the consistency of downstream quantification among different patients.

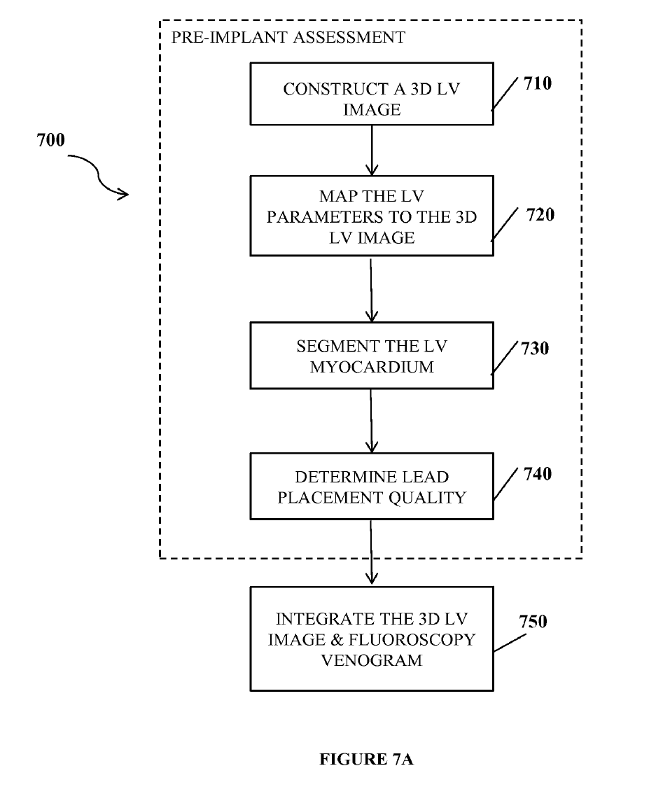

In some embodiments, the processing step 410 may include a step 412 of determining regional maximum counts and respective locations using spherical sampling (e.g., every 8° spherically) for the LV apex and cylindrical sampling (e.g., every 9° for each slice) for the rest LV. FIG. 5 shows an example of the spherical and cylindrical sampling. The processing step 410 assumes the LV apex as a hemi-sphere and the rest of the LV as a cylinder.

The processing step 410 may include a step 414 of constructing the mid-surface of the LV using the locations of the regional maximum counts. Because of the partial volume effect, the pixel with regional maximum counts should be located in the middle of the myocardial wall.

Next, the processing step 410 may include a step 416 of determining regional maximum counts and respective locations, e.g., re-sample the LV myocardium by performing spherical and cylindrical sampling, along the angles that are perpendicular to the constructed LV mid-surface. In some embodiments, the processing step 410 may further include a step 418 constructing the LV mid surface using the locations of the regional maximum counts determined in step 416.

The processing step 410, for example, steps 414 through 418, can normalize the changing size of the LV over the cardiac cycle. In some embodiments, steps 416 and 418 may be repeated until a desired normalization is achieved. For example, the steps 416 and 418 may be repeated any number of times (e.g., one time, two times, three times, more than three times, etc.) until the desired normalization is achieved.

The processing step 410, for example, as shown in FIG. 4B, can overcome deficiencies of conventional sampling techniques. The processing can construct the LV mid surface more accurately because the regional maximum counts are more accurately located. Additionally, because the processing normalizes the changing size of the LV over the cardiac cycle, the LV pixels in different temporal frames are better aligned so that the phases are more accurately calculated.

In some embodiments, the method 400 may further include a step 420 of determining the phases (also referred to as “contraction sequence”) for each sample. After the samples are collected, the samples may be approximated in the temporal domain by Fourier harmonic functions to calculate their phases (also referred to as “phase analysis”). The phase of the Fourier harmonic function for each sample may represent the onset of mechanical contraction in that myocardial region.

In some embodiments, the standard deviation of all phase values on the entire LV (i.e., phase standard deviation [PSD]) and the bandwidth including about 95% of all phase values on the entire LV (i.e., histogram bandwidth [BW]) may characterize LV mechanical dyssynchrony. Comparing the mean phase values among the myocardial segments may identify the site of latest activation (the segment with the largest mean phase value) and may characterize the LV contraction sequence (the sequence from the segment with the earliest contraction, i.e., the smallest mean phase value to the site of latest activation).

The method 400 may include a step 430 of generating the LV myocardium surface. In some embodiments, the LV myocardium surface may include the LV mid-surface. In some embodiments, the LV myocardium surface (the LV mid-surface) may be generated by the processing step 410.

In certain embodiments, the LV myocardium surface may include the LV epicardial and/or endocardial surfaces. In some embodiments, the LV epicardial and/or endocardial surfaces may be determined from the mid-surface according to any technique. In some embodiments, the LV epicardial surface may be determined by adding a predetermined thickness (e.g., 5 mm) to the mid-surface. In some embodiments, the LV endocardial surface may be determined by subtracting a predetermined thickness (e.g., 5 mm) from the mid-surface. In other embodiments, the LV epicardial and endocardial surfaces may be determined based on thresholding. For example, the generating the LV myocardium surface may include determining a pixel having a value less than a predetermined threshold (for example, 40%) along the outward for epicardial surface and along the inward for endocardial surface, perpendicular direction of each mid-surface sample; and determining the location of the pixel. The location of the pixel may represent the LV epicardial and endocardial surfaces, respectively. In certain embodiments, the LV epicardial and endocardial surfaces may be determined based on Gaussian fitting. For example, the generating the LV myocardium surface may include using a Gaussian curve to fit values of pixels that are along the perpendicular direction of each mid-surface sample; and determining the location of a threshold value, for example, the outward half-maximum point for epicardial surface and the inward half-maximum point for endocardial surface. The location of the threshold value, for example, the outward half-maximum point, may represent the LV epicardial surface; and the inward half-maximum point, may represent the LV endocardial surface.

The method 400 may include a step 440 of generating the myocardial scar distribution. “Myocardial scar” can be defined as the samples with values less than about 50% of the maximal sample value over the LV myocardium. The scar distribution may be generated according to any scar determination technique. The scar distribution may also be generated according to a different threshold or a different maximal sample value.

In some embodiments, the total number of samples defined as scar over the total number of samples on the entire LV myocardium may characterize the LV scar burden. The total number of samples defined as scar in each segment over the total number of samples in the segment may characterize the scar burden of the segment. The scar burden of all segments may characterize the distribution of scar over the LV myocardium.

In some embodiments, the step 420 of calculating each phase, the step 430 of generating the LV myocardium, the step 440 of generating the myocardial scar distribution, or some combination thereof may be performed in parallel. In other embodiments, the step 420, the step 430, the step 440, or some combination thereof may be performed in an order.

In some embodiments, the method 400 may include a step 450 of mapping the scar distribution and/or phases on top of the LV myocardial image. In some embodiments, the myocardial images including one or more quantitative indices (such as scar distribution and phases), may be outputted, for example, to a display. This may be helpful, for example, in pre-implant assessment. In some embodiments, the one or more quantitative indices, for example, related to left-ventricular function or dyssynchrony determined from myocardial images may be compared to corresponding thresholds, for example, to predict the patient response to cardiac resynchronization therapy.

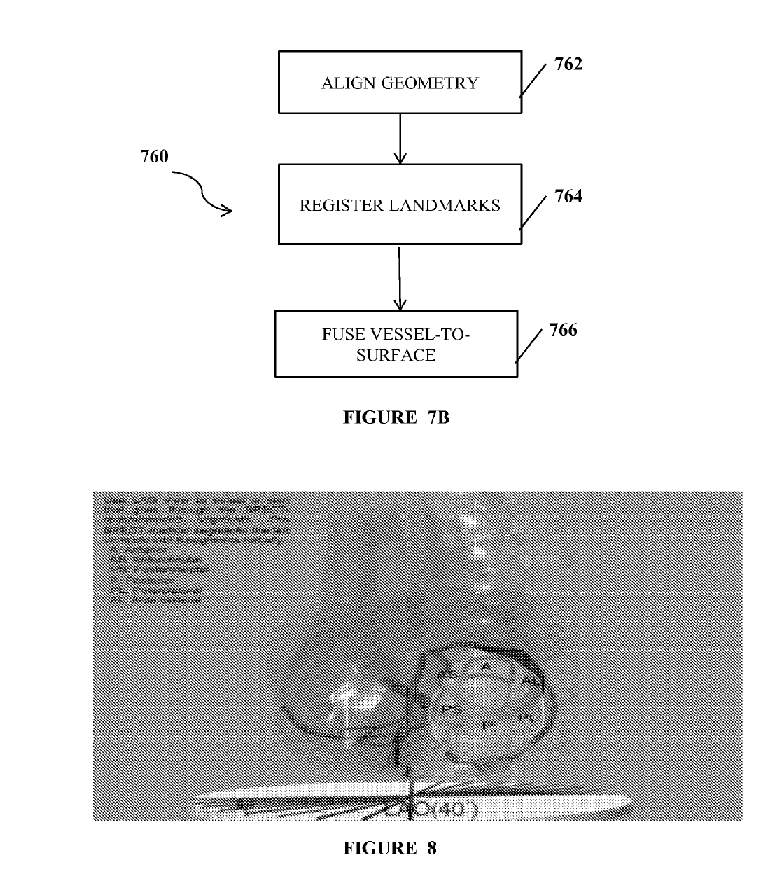

FIG. 6 shows an example of myocardial perfusion images with left-ventricular contraction sequence and scar distribution for optimal LV lead placement in cardiac resynchronization therapy.

### Determining Optimal Lead Placement/Generating Integrated Image

In some embodiments, the method 200 may include a step 240 of generating myocardial images including pre-assessment information and integrated image including the myocardial images and fluoroscopy images.

FIG. 7A shows a method 700 of generating an integrated image according to embodiments. In some embodiments, the method 700 may include a step 710 of constructing a 3D LV image using the mid-surface, epicardial surface, and/or endocardial surface of all samples determined by the method 400.

In some embodiments, the method 700 may include a step 720 of mapping the LV contraction sequence and scar distribution, calculated based on the values of the samples, on top of the 3D LV image.

In some embodiments, the method 700 may include a step 730 of segmenting the LV myocardium into a plurality of segments. The LV myocardium may be segmented into any number of segments. In some embodiments, the LV myocardium may be segmented into about thirteen segments, for example, as shown in FIG. 6. In other embodiments, the LV myocardium may be segmented into a different number of segments, for example, at least two, three, four, five, six, seven, eight, nine, ten, eleven, twelve, or more than thirteen segments.

In some embodiments, the segments may include at least one apical segment, at least one mid segment, and at least one basal segment. In some embodiments, the LV myocardium may be segmented into one apical segment, six mid segments, and six basal segments, for a total of thirteen segments, for example, as shown in FIG. 6.

In some embodiments, the step of segmenting may include dividing the LV into the apex (inner circle), the mid (mid-ring), and base (the outer ring) segments. The segmenting may further include dividing the mid and base segments into additional segments. For example, the segmenting may further segment the mid and base segments evenly into 6 segments (anterior, anterolateral, posterolateral, posterior, posteroseptal, and anteroseptal from about 12 o'clock clockwise).

In some embodiments, the method 700 may further include a step 740 of determining the lead placement quality of each segment. In some embodiments, the lead placement quality may be based on any ranking scheme. In some embodiments, the lead placement quality may include a plurality of ranks. The ranks may be numerical and/or qualitative. In some embodiments, the lead placement quality may include three different ranks. For example, the lead placement quality may include ranks indicating “optimal,” “next optimal,” and “not recommended.” In other embodiments, there may be more or less ranks. In some embodiments, the lead placement quality may be based on location and/or one more quantitative indices (e.g., scar distribution and/or contraction sequence).

In some embodiments, the determining step 740 may include determining the quality of the segments based on location. Segment(s) disposed at predetermined locations may be determined to be “not recommend.” These segments may include at least the apical segment, the mid anteroseptal segment and mid posteroseptal segment. The apical segment is generally not considered for CRT LV lead implantation because its location is close to the right-ventricular lead. The mid anteroseptal and mid posteroseptal segments are generally not considered for CRT LV lead implantation because they are hard to access.

In some embodiments, the determining step 740 may include determining the lead placement quality of each remaining segment based on one or more quantitative indices, such as scar distribution and contraction. In some embodiments, the determining step 740 may include determining the quality of each remaining segment based on scar tissue. The segments with more than 50% scar may be determined to be “not recommend” for CRT LV placement. The determining step 740 may include determining the remaining quality of each remaining segment based on the latest contraction. Among the remaining segments, the one with latest contraction (largest mean phase in the segment) may be considered to be “optimal” (displayed in red in FIG. 6) and ranked as 1. The segments near the “optimal” segment and with <50% scar may be considered to be “next-optimal” (displayed in orange in FIG. 6). According to their mean phases, the “next-optimal” segments may be ranked as 2, 3, etc. The segments that are neither “optimal” nor “next-optimal” as well as those remote from the optimal site, and labeled may be considered to be “not-recommended”.

In some embodiments, the method 700 may include a step of displaying the one or more of ranked segments on top of the 3D LV image. The one or more ranked segments on top of the 3D LV image may be a part of the pre-implant assessment. In some embodiments, only the segment(s) that are considered “optimal” may be displayed. In some embodiments, the segment(s) that are considered “optimal” or “next-optimal” may be displayed. In some embodiments, the pre-implant assessment may include one or more of the quantitative indices associated with that segment, for example, mean phase and scar burden of the segment. In some embodiments, the pre-implant assessment information may include the one or more quantitative indices and lead placement quality for one or more of the segments. The segment may be displayed with the pre-assessment information on the myocardial image (3D LV image).

In some embodiments, the method 700 may include a step 750 of integrating the myocardial image (3D LV image with one or more ranked segments) with the fluoroscopy venogram. In some embodiments, the integrating may include translating, rotating, and zooming the 3D LV images to fuse the 3D LV images with the 2D fluoroscopy venogram.

In some embodiments, the 3D LV images may be integrated with 3D venous anatomy, for example, processed or reconstructed from the 2D fluoroscopy venogram image data (e.g., step 222). FIG. 7B shows a method 760 of integrating the myocardial image with the reconstructed 3D venous anatomy, according to embodiments. It will be understood that the integrating step 750 may be performed by applying other sampling techniques.

FIG. 7B shows a method of integrating the epicardial surface extracted from the myocardial image, for example, determined in step 430, with the reconstructed 3D venous anatomy, for example, determined in step 222.

As shown in FIG. 7B, the method 760 may include a step 762 of aligning the geometry based on the fluoroscopy venogram and myocardial images. The step 762 may include aligning the 3D venous anatomy (reconstructed from the fluoroscopy venograms) with the epicardial surface extracted from myocardial images using the geometric parameters provided by these imaging modalities. The geometric parameters may include but is not limited to left-ventricular centers and angles, isocenters and magnification factors.

The aligning step 762 may include comparing the geometric parameters of the myocardial image with those of the fluoroscopy image to calculate a transformation matrix. The transformation matrix may consist of parameters. In some embodiments, the parameters may be about six parameters (three for translation, and three for rotation). The transformation matrix may then be applied to the 3D venous anatomy to translate and rotate it in 3D space, so that its spatial position becomes similar to that of the epicardial surface.

After the aligning step 762, spatial positions of the epicardial surface and the 3D venous anatomy can generally correspond to the same spatial coordinates. In other words, the transformation matrix calculated and used in aligning step 762 can change the spatial coordinates of the 3D venous anatomy to the spatial coordinates that in the same spatial system with the epicardial surface.

Next, the method 760 may include a step 764 of registering the landmarks of the 3D venous anatomy and epicardial surface. The registering step 764 can reduce any misalignment between the 3D venous anatomy and epicardial surface after the aligning step 762. The misalignment can be due to different patient positions during the fluoroscopy and myocardial imaging. In some embodiments, the registering step 764 can redefine a position of the 3D venous anatomy with respect to the myocardial image.

The registering step 764 may include registering the anterior vein to the anterior interventricular groove, the middle cardiac vein to the posterior interventricular groove, and/or coronary sinus to LV posterolateral base by a least-square minimization of distances between pixel pairs established in the aligned 3D venous anatomy and epicardial surface. The grooves and base can be identified on the myocardial image during processing. The registration may be performed using rigid registration techniques.

The registering step 764 can be implemented to handle the differences in length between the reconstructed veins and grooves. Generally, the reconstructed anterior vein and middle cardiac vein from the fluoroscopy venogram can have different lengths than the identified inter-ventricular grooves on the epicardial surface of the myocardial images.

The registering step 764 can accurately register the images despite these differences. In some embodiments, the registering step 764 may include the following steps: 1) extending the inter-ventricular grooves to find their intersections with the valve plane on the epicardial surface; 2) corresponding the intersections between the extended grooves and valve plane with the beginnings of the reconstructed anterior vein and middle cardiac vein, respectively; 3) establishing corresponding pixel pairs along the extended inter-ventricular grooves, anterior vein and middle cardiac vein; 4) establishing corresponding pixel pairs of the coronary sinus (on the 3D venous anatomy) and posterolateral base (on the epicardial surface); and 5) least-square minimizing the distances among the corresponding pixel pairs to register the 3D venous anatomy and the epicardial surface.

The least-square minimizing includes calculating a transformation matrix. The transformation matrix may consist of parameters. In some embodiments, the parameters may be about six parameters (three for translation, and three for rotation). When the transformation matrix is applied to the 3D venous anatomy to translate and rotate it in 3D space, the sum of the square of the distances among the corresponding pixel pairs can be minimized The transformation matrix can be applied to the 3D venous anatomy to register it with the epicardial surface. The transformation matrix calculated and used in registering step 764 may refine the spatial position of the 3D venous anatomy, which has already be converted to the same spatial system with the epicardial surface in the aligning step 762.

Following the registering step 764, the method 760 may include a step 766 of fusing (overlaying) the 3D venous anatomy onto the epicardial surface using the spatial position of the 3D venous anatomy determined in the registered image 764. The fusing step 766 can address any misalignment on the surface, e.g., for example, the veins may not be completely or correctly overlaid on the epicardial surface. The fusing step can modify the registered image by overlaying the 3D venous anatomy onto an epicardial surface provided in the myocardial image. The fusing step 766 thus can improve the registered and aligned image.

In some embodiments, the fusing step 766 may include 1) distance minimization or 2) cylindrical projection. The distance minimization can determine the pixels on the epicardial surface, which have closest distances to the corresponding pixels on the 3D venous anatomy. The cylindrical projection can connect the pixels on the 3D venous anatomy and the centers of the left ventricle on the same short-axis view, and then find the intersections between the connective lines and the epicardial surface. Those intersections can be then linked into the cylindrically projected lines on the epicardial surface, which correspond to the 3D venous anatomy. The cylindrically projected lines can represent the 3D venous anatomy overlaid on the epicardial surface.

In some embodiments, after fusing the images, the pre-assessment information may be included with the integrated image. For example, the integrated image may indicate the optimal segment for LV lead placement and/or associated one or more quantitative indices, such as scar distribution and contraction sequence. In some embodiments, the integrated image may indicate the rank of one, some, or each segment. In some embodiments, the integrated image may indicate the rank of the “optimal” and “next-optimal” segments. This image may be used to guide LV lead implantation in cardiac resynchronization therapy.

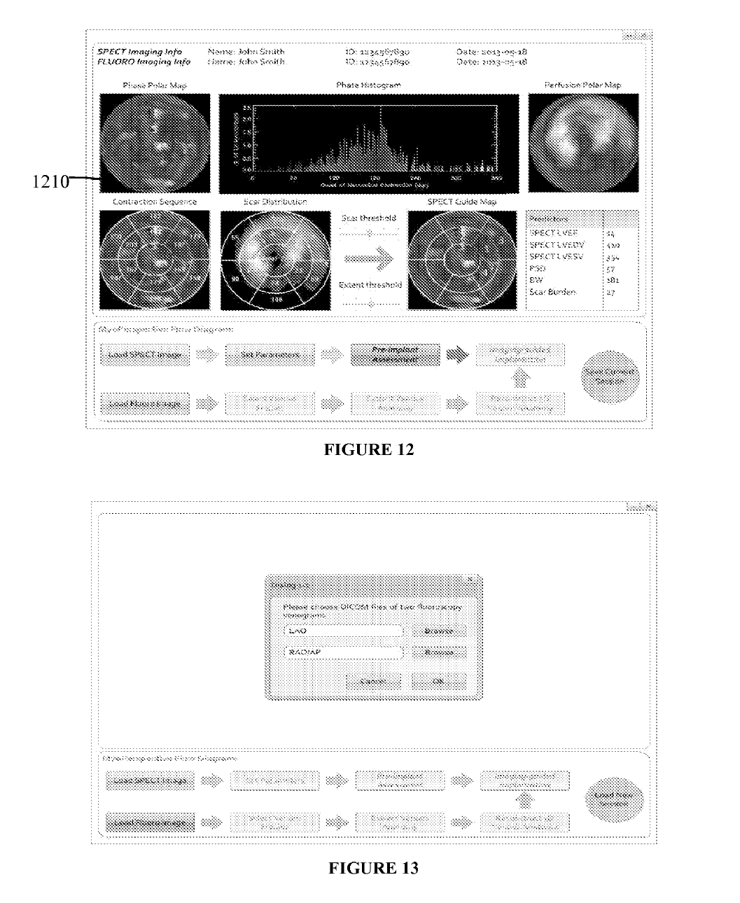

In some embodiments, the rank may identified by a numerical, symbolic, alphameric and/or color marker. The marker may be any symbol. FIG. 8 shows an example of an integrated image that includes 2D venous anatomy and myocardial perfusion image.

### Outputting

In some embodiments, the method may further include a step 250 of outputting the generated image and/or pre-assessment information. In some embodiments, the outputting may include displaying, printing, storing, and/or transmitting the generated image. In some embodiments, the integrated image may be transmitted to another system, server and/or storage device for the printing, displaying and/or storing the generated image.

In some embodiments, the method may further include displaying at least one parameter or quantitative index related to a region or segment of the generated image. The parameter may relate to features of the heart. The region may be selected by the operator or may be displayed based on a position of an interventional device. The interventional device may be any device used for cardiac intervention procedures. The interventional device may include but is not limited to a probe, a catheter, and an ablation device.

In some embodiments, the method may further include transmitting the generated image and/or pre-assessment information to another system. In some embodiments, the method may further include transmitting the generated image and/or pre-assessment information to an interventional system. The interventional system may be any system configured for cardiac interventional procedures. The method may further include displaying a position of an interventional device within the heart on the generated image. The method may further include displaying specific pre-assessment information, such as parameters and/or quantitative indices, of a selected region or segment of the heart.

In some embodiments, the generated image may be used for planning and/or during a cardiac interventional procedure, such as implanting a CRT device.

The outputting step 250 may individually or consequentially output the generated images. The generated images include but are not limited to the generated integrated image and/or the processed myocardial image. For example, the myocardial image, for example, after step 450 and/or step 740, may be outputted separately from the integrated image.

In some embodiments, the steps of the methods may be performed over a wired, wireless, or combination thereof In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radio frequency network, or another similarly functioning wireless network.

### System Implementation

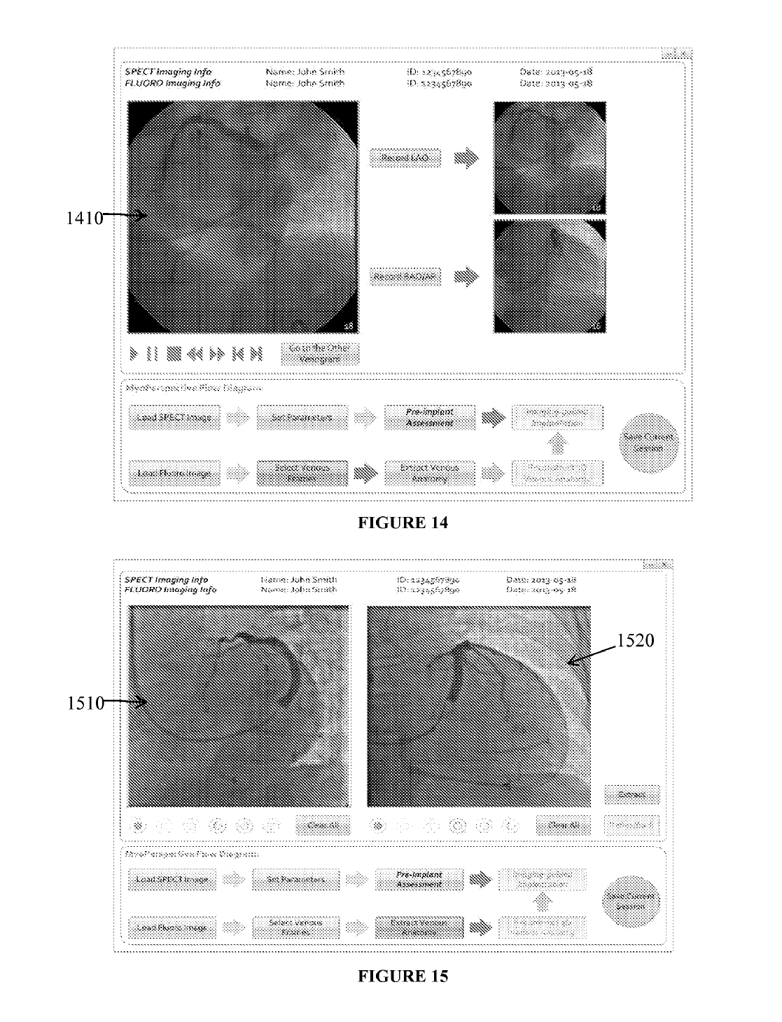

FIG. 9 shows an example of a system that may be used to generate an integrated image according to embodiments. The system 900 may include any number of modules that communicate with other through electrical or data connections (not shown). In some embodiments, the modules may be connected via a wired network, wireless network, or combination thereof In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radio frequency network, or another similarly functioning wireless network.

Although the modules of the system are shown as being directly connected, the modules may be indirectly connected to one or more of the other modules of the system. In some embodiments, a module may be only directly connected to one or more of the other modules of the system.

It is also to be understood that the system may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the system may be time synchronized. In further embodiments, the system may be time synchronized with other systems, such as those systems that may be on the medical facility network.

As shown in FIG. 9, the system 900 may optionally include at least two patient imaging systems 910 and 920. The medical imaging device 910 may be any imaging system configured to obtain and generate myocardial images (e.g., 3D left-ventricular myocardial images with identifiable inter-ventricular grooves). The medical imaging device 910 may include but is not limited to SPECT imaging system, PET, MRI, cardiac CT, or echocardiography. The medical imaging system 920 may be any fluoroscopic imaging system capable generating fluoroscopic venograms. In other embodiments, the system 900 may communicate with the imaging systems and/or a data storage device.

In some embodiments, the medical imaging systems 910 and 920 may include a computer system to carry out the image processing. The computer system may further be used to control the operation of the system or a separate system may be included.

The system 900 may further include a computing system 930 capable of generating the integrated image and/or pre-assessment information. In some embodiments, the computing system 930 may be a separate device. In other embodiments, the computing system 930 may be a part (e.g., stored on the memory) of other modules, for example, the interventional device 980 or one or both imaging devices 910 and 920, and controlled by its respective CPUs.

The system 930 may be a computing system, such as a workstation, computer, or the like. The system 930 may include one or more processors 932. The processor 932 may be one or more of any central processing units, including but not limited to a processor, or a microprocessor. The processor 932 may be coupled directly or indirectly to one or more computer-readable storage medium (e.g., physical memory) 944. The memory elements, such random access memory (RAM), read only memory (ROM), disk drive, tape drive, etc., or a combinations thereof The memory may also include a frame buffer for storing image data arrays. The memory 932 may be encoded or embed with computer-readable instructions, which, when executed by one or more processors 932 cause the system 930 to carry out various functions.

In some embodiments, the disclosed methods (e.g., FIGS. 1-4 and 7) may be implemented using software applications that are stored in a memory and executed by a processor (e.g., CPU) provided on the system. In some embodiments, the disclosed method s may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system. In addition, various other peripheral devices may be connected to the computer platform such as an additional data storage device, a printing device, and other I/O (input/output) devices.

In some embodiments, the system 930 may include an image processor 934 configured to process myocardial image data and/or fluoroscopy venogram image data. In some embodiments, the image processor 934 may be configured to process the 2D fluoroscopy venogram image data to generate 3D venous anatomy. In some embodiments, the system 900 may further include an image quantifier device 936 configured to quantify the processed myocardial image to determine one or more quantitative indices (e.g., LV systolic and diastolic function or dyssynchrony). In some embodiments, the system 900 may further include an image generator 946 configured to generate the processed myocardial image with pre-implant assessment information and/or an image with integrated fluoroscopic venogram and 3D LV images, at least one of the images indicating the quality of at least one segment for LV lead placement.

In some embodiments, the system 930 may include a communication interface 948 configured to conduct receiving and transmitting of data between other modules on the system and/or network. The communication interface 948 may be a wired and/or wireless interface, a switched circuit wireless interface, a network of data processing devices, such as LAN, WAN, the internet, or combination thereof The communication interface may be configured to execute various communication protocols, such as Bluetooth, wireless, and Ethernet, in order to establish and maintain communication with at least another module on the medical facility network.

In some embodiments, the system 900 may include an input/output 938 configured for receiving information from one or more input devices 950 (e.g., a keyboard, a mouse, and the like) and/or conveying information to one or more output devices 960 (e.g., a printer, a CD writer, a DVD writer, portable flash memory, etc.).

In some embodiments, the system 900 may include one or more input devices 950 configured to control the generation of the medical images, display of medical images on a display, and/or printing of the images by a printer interface. The input devices 950 may include a mouse, joystick, keyboard, track ball, touch activated screen, light wand, voice control, or any similar or equivalent input device, and may be used for interactive geometry prescription.

In some embodiments, the system 900 may include graphics controller 942 configured to process data for presentation on a monitor, such as display 970, in a human readable format.

In some embodiments, the system 900 may include or communicate with an interventional device or system 980 configured for cardiac interventional procedures. The system may be any system, and may be capable for CRT lead placement.

In some embodiments, the system 900 may include one or more data repositories 990. The data repositories 990 may be configured to store the raw and/or processed images.

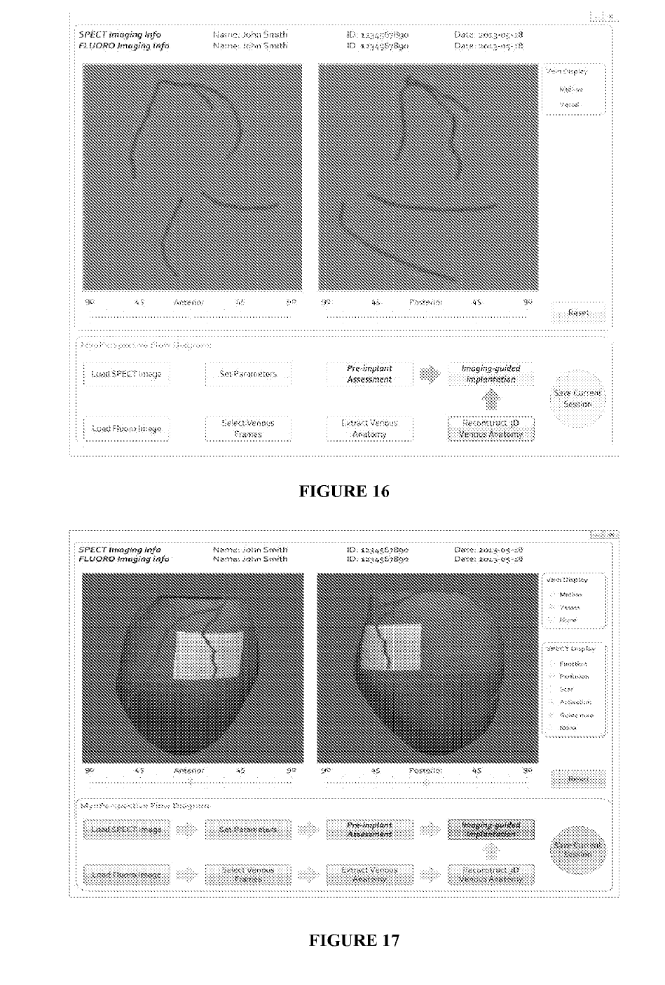

FIGS. 10-17 show conceptual diagrams show an example of an implementation of the methods, computer-readable media, and systems to process myocardial and fluoroscopy venogram image data and generate images according to embodiments. The diagrams show a MyoPerspective user interface 1000 that may be displayed on a display, for example, display 970 that may be used in conjunction with and/or implemented on the computing system 930, alone and/or a part (e.g., stored on the memory) of other modules, for example, the interventional device 980 or one or both imaging devices 910 and 920, and controlled by its respective CPUs.

In some embodiments, the user interface 1000 may be capable of at least: 1) processing myocardial images for pre-implant assessment; 2) processing fluoroscopy images to obtain 3D venous anatomy; and/or 3) integrating the myocardial images and the fluoroscopy venograms, for example, for imaging-guided implantation.

In some embodiments, the user interface 1000 can be capable of reducing intraoperative time. For example, the myocardial images may be acquired a few days before the CRT procedure. In this way, the myocardial image processing and the pre-implant assessment can be performed prior to the CRT procedure.

In some embodiments, the user interface 1000 may include a workflow 1010 of the functions, as shown in FIG. 10. In other embodiments, the workflow 1010 may be omitted.

As shown in FIG. 10, the work flow 1010 may begin with loading the SPECT (i.e., myocardial perfusion) image data. After which, the data may be prepared for processing. FIG. 11 shows an example of the loaded SPECT (myocardial perfusion) image data 1110 for processing.

In some embodiments, a user may be prompted to manually input one or more parameters for processing the SPECT image data to generate a myocardial perfusion image with at least left-ventricular contraction sequence and scar distribution. The parameters may include but are not limited to the following:
i. center and radius of the left ventricle;
ii. center-short axis slice of the left ventricle;
iii. apical short-axis slice of the left ventricle;
iv. basal short-axis slice of the left ventricle;
v. apical short-axis slice of the right ventricle;
vi. basal short-axis slice of the right ventricle;
vii. anterior inter-ventricular groove point on the apical short axis slice of the right ventricle;
viii. anterior inter-ventricular groove point on the basal short axis slice of the right ventricle;
ix. posterior inter-ventricular groove point on the apical short axis slice of the right ventricle; and/or
x. and posterior inter-ventricular groove point on the basal short axis slice of the right ventricle.

In the example show in FIG. 11, the following parameters were selected:
i. center and radius of the left ventricle of (32, 32, 7);
ii. center-short axis slice of the left ventricle of (31);
iii. apical short-axis slice of the left ventricle of (23);
iv. basal short-axis slice of the left ventricle of (40);
v. apical short-axis slice of the right ventricle of (33);
vi. basal short-axis slice of the right ventricle of (35);
vii. anterior inter-ventricular groove point on the apical short axis slice of the right ventricle of (20, 16);
viii. anterior inter-ventricular groove point on the basal short axis slice of the right ventricle of (20, 16);
ix. posterior inter-ventricular groove point on the apical short axis slice of the right ventricle of (18, 42); and/or
x. posterior inter-ventricular groove point on the basal short axis slice of the right ventricle of (16, 44).

In other embodiments, the parameters may be pre-stored or automatically determined

FIG. 12 shows an example of the processed SPECT image data 1210 based on the parameters provided above. The SPECT image data may be processed, for example, according to the method shown and described with respect to FIGS. 4-7, to determine pre-implant assessment information. The pre-implant assessment information may include one or more of the quantitative indices, lead quality, other parameters, or any combination thereof The pre-implant assessment information may include but is not limited to phase polar map, phase histogram, perfusion polar map, contraction sequence, scar distribution, and/or guide map. The phase polar map may show the distribution of onset of mechanical contraction of left-ventricular myocardial regions. The phase histogram may show the left-ventricular global mechanical dyssynchrony. The perfusion polar map may show left-ventricular myocardial perfusion. The contraction sequence may show the sequence of the left-ventricular myocardial contraction in a segment model (e.g., a 13-segment model). The scar distribution may show the sequence of the left-ventricular myocardial scar in a segment model (e.g., a 13-segment model). The SPECT guide map may combine the assessment of the contraction sequence and scar distribution and show the optimal and suboptimal segments for left-ventricular lead placement.

In some embodiments, the user interface 1000 may include predictors. The predictors may be quantitative indices related to the left-ventricular function and dyssynchrony. The predictors may be useful to predict the patient response to cardiac resynchronization therapy.

Next, as shown in FIG. 13, the fluoroscopy venogram image data may be loaded and processed before the integrated image may be generated. In some embodiments, the fluoroscopy venogram image data acquired to guide the lead placement may be used. The fluoroscopy image data 1410 may include two fluoroscopy images acquired from different angles, as shown in FIG. 14. This image data are conventionally acquired and performed in current CRT procedure practice. In this way, the user interface 1000 may be used interoperatively.

In some embodiments, after loading the fluoroscopy venograms, a user may be prompted to manually select a frame from each venogram for processing. The selected frame may be a frame that best shows left-ventricular venous anatomy. In other embodiments, the frame may be automatically selected.

Next, as shown in FIG. 15, the left-ventricular veins may be identified. In some embodiments, the veins may be manually identified by the user. The venous identification includes drawing lines along each vein on each frame on a user graphic interface of computer software and indicating the correspondences of the lines. For example, in FIG. 15, the lines labeled “5” on the two frames 1510 and 1520 correspond to the same vein, i.e., the middle cardiac vein. Accurate correspondence cannot not only required for pairing the veins between the two frames, but also for pairing the venous segments along each vessel, which is important for the 3D reconstruction.

In some embodiments, the venous identification may be at least partially automated, for example, to reduce the processing time. In some embodiments, the topology of the left-ventricular venous anatomy can be displayed to help the user quickly determine the corresponding vessels and draw lines accordingly. In some embodiments, neighboring vessel search can be used to search the vessels near the drawn lines. This can shorten the processing for venous identification, because it can allow the user to quickly and roughly draw the lines near the vessels and then automatically find the actual vessels on the fluoroscopy frames. In some embodiments, edge detection may be used to improve the segmental correspondences and the accuracy of 3D reconstruction.

After the left-ventricular veins are identified on each fluoroscopy frame, the image data may be processed to reconstruct the 3D venous anatomy, for example, as shown in FIG. 16.

Next, the integrated image may be generated. In some embodiments, the integration of the 3D SPECT epicardial surface and the left-ventricular venous anatomy may be automatic to generate the integrated image. The image may be integrated by geometric alignment, landmark registration, and vessel-to-surface fusion. The geometric alignment may include aligning the 3D venous anatomy with the epicardial surface using the geometric parameters provided by the images. The landmark registration may include rigid registering the anterior vein to the anterior inter-ventricular groove and the middle cardiac vein to the posterior interior-ventricular groove by least-square minimization distance. The grooves may be identified on the SPECT image when setting the parameters for the SPECT image processing (FIG. 11). The vessel-to-surface fusion may include fusing the venous anatomy onto the SPECT epicardial surface by least-square minimization of distance. The fusion may generate the integrated image.

FIG. 17 shows the generated integrated image (SPECT-Vein fusion). As shown in FIG. 17, at least one view of the generated integrated image may be displayed. In some embodiments, two different views 1710 and 1720 of the image may be displayed. In some embodiments, the user can be allowed to rotate the image. In some embodiments, the generated integrated image may also include pre-assessment information. The generated image may also identified one segment 1712 considered to be “optimal” for lead placement. The vein 1714 related to that segment 1712 may also be displayed.

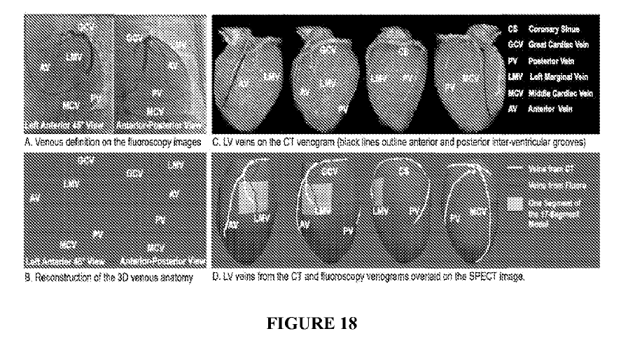

FIG. 18A-D show results of patient image data processed using the MyoPerspective user interface 1000. FIG. 18A show major left-ventricular veins that were manually identified on two selected fluoroscopy frames with clear venous anatomy. FIG. 18B show the reconstructed frames into a 3D anatomy. FIG. 18D show the integrated image (reconstructed 3D venous anatomy fused with the epicardial surface on SPECT). FIG. 18C show the post-implant CT venogram. The CT was fused with SPECT epicardial surface and compared to the locations of the veins given by the fluoroscopy (gray lines) and CT images (white lines) shown in FIG. 18D.

The distances of their corresponding venous segments were 2.7±7.1 mm (range: 0.3-16.4 mm), much smaller than the segmental size of the 13-segment model (usually >30×30 mm²) D displays an anterolateral segment with the implanted LV lead. The left marginal veins from CT and fluoroscopy are generally located in the same segment, when fused with the SPECT image. Thus, the MyoPerspective user interface can provide an acceptable spatial accuracy for guiding left-ventricular lead placement.

It is to be understood that the embodiments of the disclosure may be implemented in various forms of hardware, software, firmware, special purpose processes, or a combination thereof In one embodiment, the disclosure may be implemented in software as an application program tangible embodied on a computer readable program storage device. The application program may be uploaded to, and executed by, a machine comprising any suitable architecture. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

While the disclosure has been described in detail with reference to exemplary embodiments, those skilled in the art will appreciate that various modifications and substitutions may be made thereto without departing from the spirit and scope of the disclosure as series forth in the appended claims. For example, elements and/or features of different exemplary embodiments may be combined with each other and/or substituted for each other within the scope of this disclosure and appended claims.

What is claimed is:

1. A method for generating an integrated image, comprising:
processing a myocardial perfusion image to determine left ventricle (LV) systolic and diastolic function, the quantifying including generating myocardial scar distribution; and
generating an integrated image including myocardial perfusion image and fluoroscopy venogram data, the integrated image including at least one rank of lead placement quality.

2. The method according to claim 1, wherein the generating includes segmenting the myocardial perfusion image into a plurality of segments, and determining lead placement quality of each segment based on location, scar distribution, and contraction sequence.

3. The method according to claim 1, wherein the image includes a plurality of ranks, the ranks including qualitative and/or numerical ranks.

4. The method according to claim 3, the ranks including “optimal,” “next optimal,” and/or “not recommended.”

5. The method according to claim 1, wherein the generating includes segmenting the myocardial perfusion image data, the segmenting including dividing the myocardial perfusion image data into a plurality of location-based segments.

6. The method according to claim 5, wherein the segments include apex, mid and/or base segments.

7. The method according to claim 1, further comprising displaying the integrated image with the lead placement quality.

8. The method according to claim 1, further comprising transmitting the integrated image to a cardiac interventional system.

9. The method according to claim 1, further comprising receiving post-processed myocardial perfusion image data.

10. The method according to claim 1, wherein the myocardial perfusion image data is a SPECT image data.

11. The method according to claim 1, further comprising:
wherein the processing quantifies the myocardial perfusion image data.

12. The method according to claim 1, wherein the processing includes:
determining regional maximum counts and respective locations along one or more angles that are perpendicular to a constructed LV mid-surface; and
constructing mid-surface of the LV mid surface using the locations of the regional maximum counts.

13. A computer-readable storage medium storing instructions for generating an integrated image, the instructions comprising:
processing SPECT image data to determine LV systolic and diastolic function, the processing including generating myocardial scar distribution; and
generating an integrated image including SPECT image data and fluoroscopy venogram data, the integrated image including at least one rank of lead placement quality.

14. A system for generating an integrated image, comprising:
an image quantifier configured to process SPECT image data to determine LV systolic and diastolic function, the processing including generating myocardial scar distribution and LV contraction sequence; and
an integrated image generated configured to generate an integrated image including SPECT image data and fluoroscopy venogram data, the integrated image including at least one rank of lead placement quality.
